(19) United States
(12) Patent Application Publication
(10) Pub. No.: US 2024/0422884 A1
(43) Pub. Date: Dec. 19, 2024

(54) LIGHTING SYSTEMS AND APPLICATIONS THEREOF

(71) Applicant: Midwest Lighting Institute, Inc., Cottage Grove, WI (US)

(72) Inventors: Rodney Heller, Cottage Grove, WI (US); Brian Liebel, West Palm Beach, FL (US)

(21) Appl. No.: 18/740,906

(22) Filed: Jun. 12, 2024

Related U.S. Application Data
(60) Provisional application No. 63/507,837, filed on Jun. 13, 2023.

(51) Int. Cl. H05B 47/16; A61N 5/06

(52) U.S. Cl. CPC H05B 47/16; A61N 5/0618; A61N 2005/0663

(57) ABSTRACT

Provided herein are lighting devices, systems, and methods. In particular, provided herein are lighting systems configured for use in a variety of facilities or settings to improve the health, performance, and well-being of one or more individuals or groups of people that reside in, work in, or visit the facility or setting.

The present application claims priority to U.S. Provisional Patent Application Ser. No. 63/507,837, filed Jun. 13, 2023, the disclosure of which is herein incorporated by reference in its entirety.

### FIELD

Provided herein are lighting devices, systems, and methods. In particular, provided herein are lighting systems configured for use in a variety of facilities or settings to improve the health, performance, and well-being of one or more individuals or groups of people that reside in, work in, or visit the facility or setting.

### BACKGROUND

Light, in the form of fire, was first harnessed by mankind 125,000 years ago. It slowly advanced through stages of fire for light until 1800, when Humphry Davy invented the first arc lamp based on electrical current from batteries. Then in 1879, Thomas Edison and Joseph Swan patented the first carbon thread incandescent lamp. Since that first patent by Edison, man-made lighting has advanced through fluorescents, high pressure sodium and now the LED revolution. All of these advances were based on more lumens/watt or better light solely for visual tasks. Vision is enabled by two photoreceptors, the rods and cones. In 1999 Samar Hattar discovered a third photoreceptor, the ipRGC. This is a non-visual photoceptor in mammals responsible for entraining the circadian clock, influencing mood, learning, vision, and potentially a host of yet undiscovered functions.

Manmade lighting environment includes, for example, healthcare facilities, nursing homes, assisted living facilities, correctional institutions, mental health facilities, schools, universities, and office buildings. Existing lighting system in many facilities are designed primarily or entirely to provide visible light and/or aesthetics. Such lighting can be suboptimal when considering other dimensions, such a the health and well being of subjects residing within, working with, or using such facilities. There are a number of challenges that exist in many facilities that can be exacerbated by improper lighting. For example, health care facilities such as hospitals and nursing facilities have abundant challenges associated with the well being of patients and workers.

A skilled nursing facility provides specialized care and rehabilitation services to patients following a hospital stay. There are approximately 18,700 skilled nursing facilities nationwide in the United States, and about 80 percent of them are also certified as nursing homes, which provide longer-term care. About 40 percent of people over age 65 will spend time in a nursing home at some point.

As hospitals have moved to shorten patient stays, skilled nursing care has grown dramatically. Medicare spending on skilled nursing facilities more than doubled to $26 billion between 2000 and 2010. About one-in-five Medicare patients who were hospitalized in 2011 spent time in a skilled nursing facility.

One-in-three patients in skilled nursing facilities suffered a medication error, infection or some other type of harm related to their treatment, according to a 2014 report by the U.S. Department of Health and Human Services (HHS) (February 2014; OEI-06-11-00370). Skilled nursing care is defined as treatment in nursing homes for up to 35 days after a patient was discharged from an acute care hospital. Doctors who reviewed the patients' records determined that 59 percent of the errors and injuries were preventable. More than half of those harmed had to be readmitted to the hospital at an estimated cost of $208 million for the month studied, about 2 percent of Medicare's total inpatient spending. The doctors found that 22 percent of patients suffered events that caused lasting harm, and another 11 percent were temporarily harmed. In 1.5 percent of cases the patient died because of poor care, the report said. Though many who died had multiple illnesses, they had been expected to survive.

The injuries and deaths were caused by substandard treatment, inadequate monitoring, delays or the failure to provide needed care, the study found. The deaths involved problems such as preventable blood clots, fluid imbalances, excessive bleeding from blood-thinning medications and kidney failure.

Fatigue of care providers, such as nurses or doctors is a factor in nursing homes and other facilities and can contribute to errors. Workload, work hours, work structures, and many other factors can indirectly or directly cause fatigue in multiple industries and affect safety.

Within the healthcare setting one of today's greatest challenges is delivering safer care in complex, fast-moving environments. Adverse events occur, and unintentional but serious harm comes to patients during routine clinical practice or as a result of a clinical decision. Fatigue is a factor that has been linked to stress, safety, and performance decrements in numerous work environments (Leung, Chan, Ng, & Wong, Applied Ergonomics, 37 (5), 565-576 2006).

Furthermore, many patient environments within health-care facilities are one-size-fits all. These present non-ideal healing and recovery environments for many patients. Additional methods for reducing errors and improving patient and staff well-being are needed.

In the US, falls are the leading cause of injury-related deaths for those 65 or older. The economic impact of falls is estimated at $754 million for fatal falls, and $50 billion for nonfatal falls. Furthermore, the US population is aging, and the number of elderly people residing in long-term care settings is increasing. An abundance of data demonstrates that falls can be prevented, but instead, the mortality rate of falls continues to rise. Current solutions to prevent falls are resource and time heavy, including expensive and burdensome infrastructure changes, expensive additional staffing, and/or active and burdensome educational courses for care providers. A new, passive, low-cost, low-burden solution is needed to decrease the number of falls sustained by the elderly.

### SUMMARY

Provided herein are lighting devices, systems, and methods. In particular, provided herein are lighting systems configured for use in a variety of facilities or settings to improve the health, performance, and well-being of one or more individuals or groups of people that reside in, work in, or visit the facility or setting. Experiments conducted during the development of the technologies provided herein revealed that particular lighting systems could not only avoid causing harm or causing reduction in well-being to people within facilities, but could significantly improve health, well-being, and performance.

For example, in some embodiments, provided herein are systems and methods for preventing adverse patient or staff events (e.g., patient falls) in a facility (e.g., care home), comprising: exposing subjects in a facility (e.g., care home) to a lighting system that: a) in patient rooms, provides blue-enriched white light during daytime hours and low-intensity, blue-depleted white light during nighttime hours; b) in common areas, provides graduated high-intensity, blue-enriched white light during daytime hours, low-intensity, blue-depleted white light during evening hours, and lower-intensity, blue depleted white light during nighttime hours; and/or c) in activity areas, provide high-intensity, blue-enriched white light during activity hours. In some embodiments, the facility is a care home. In some embodiments, the care home is a healthcare facility, nursing home, or assisted living facility.

In some embodiments, provided herein are methods for improving resident well-being and staff performance in a facility, comprising: exposing residents and staff in a facility to a lighting system that: a) in resident rooms, provides blue-enriched white light during daytime hours and low-intensity, blue-depleted white light during nighttime hours; b) in common areas, provides graduated high-intensity, blue-enriched white light during daytime hours, low-intensity, blue-depleted white light during evening hours, and lower-intensity, blue depleted white light during nighttime hours; and c) in activity areas, provide high-intensity, blue-enriched white light during activity hours; and d) in staff areas, provide high-intensity, blue-enriched while light during all hours, when occupied. In some embodiments, the facility comprises a healthcare facility, a nursing home, an assisted living facility, a correctional institution, and a mental health facility.

In some embodiments, the daytime hours are 6 AM to 6 PM, the evening hours are 6 PM to 9 PM, and the nighttime hours are 9 PM to 6 AM. The invention is not limited to these specific time boundaries. For example, in some embodiments, the daytime hours are 7 AM to 6 PM, the evening hours are 6 PM to 9 PM, and the nighttime hours are 9 PM to 7 AM. In some embodiments, the daytimes hours are 7 AM to 6 PM, the evening hours are 6 PM to 10 PM, and the nighttime hours are 10 PM to 7 AM.

In some embodiments, the patient rooms are provided at least five distinct lighting periods: i) an early morning period, ii) a mid-morning and afternoon period, iii) an early evening period, iv) an evening period, and v) a bedtime period. In some embodiments, the light fixtures in the patient rooms generate a minimum of 40 mel-EDI during the early morning period. In some embodiments, the light fixtures in the patient rooms generate a minimum of 100 mel-EDI during the mid-morning and afternoon period. In some embodiments, the light fixtures in the patient rooms generate a maximum of 40 mel-EDI during the early evening period. In some embodiments, the light fixtures in the patient rooms generate a maximum of 20 mel-EDI during the evening period. In some embodiments, the light fixtures in the patient rooms generate a maximum of 10 mel-EDI during the bedtime period, under control of a patient. In some embodiments, under medical staff control, the patient rooms can switch during the bedtime period, to generate a maximum of 100 mel-EDI.

In some embodiments, the common areas comprise hallways. In some embodiments, the hallways are adjacent to patient rooms. In some embodiments, the common areas are provided at least six distinct lighting periods: i) an early morning period, ii) a mid-morning period, iii) an afternoon period, iv) an early evening period, v) an evening period, and vi) a bedtime period. In some embodiments, the light fixtures in the common areas generate a minimum of 100 mel-EDI during the early morning period. In some embodiments, the light fixtures in the common areas generate a minimum of 135 mel-EDI during the mid-morning period. In some embodiments, fixtures in the common areas generate a minimum of 100 mel-EDI during the afternoon period. In some embodiments, the light fixtures in the common areas generate a minimum of 60 mel-EDI during the early evening period. In some embodiments, the light fixtures in the common areas generate a minimum of 30 mel-EDI during the evening period. In some embodiments, the light fixtures in the common areas generate a maximum of 10 mel-EDI during the bedtime period.

In some embodiments, the activity areas comprise dining areas and recreational activity areas. In some embodiments, the light fixtures in the dining areas generate a minimum of 135 mel-EDI when occupied and are otherwise off. In some embodiments, the light fixtures in the recreational activity areas generate a minimum of 150 mel-EDI when occupied and are otherwise off.

In some embodiments, the facility further comprises staff areas. In some embodiments, the staff areas provide high-intensity, blue-enriched white light. In some embodiments, the staff areas comprise nursing stations/control rooms and staff break rooms. In some embodiments, light fixtures in the nursing stations/control rooms generate a minimum of 150 mel-EDI at all time periods. In some embodiments, light fixtures in the staff break rooms generate a minimum of 250 mel-EDI when occupied.

In some embodiments, the lighting systems are automated lighting systems.

Also provided herein are lighting systems configured to control light in patient rooms, common areas, activity areas, and staff areas so as to effectuate any of the methods described herein.

Other aspects of the disclosure will become apparent by consideration of the detailed description and accompanying drawings.

### BRIEF DESCRIPTION OF THE DRAWINGS

FIG. 1 shows exemplary layouts and specifications of patient rooms. FIG. 1A shows a first exemplary configuration. FIG. 1B shows a second exemplary configuration.

FIG. 2 shows exemplary layouts and specifications of staff areas. FIG. 2A shows an exemplary configuration of a staff room. FIG. 2B shows a first exemplary configuration of a nurse's station. FIG. 2C shows a second exemplary configuration of a nurse's station.

FIG. 3 shows exemplary layouts and specifications of common areas. FIG. 3A shows a first exemplary hallway configuration. FIG. 3B shows a second exemplary hallway configuration. FIG. 3C shows an exemplary prison hallway configuration. FIG. 3D shows an exemplary dining or activity room configuration.

FIG. 4 shows an exemplary layout and specification of a prison cell.

FIG. 5 shows illumination data from multiple sites using embodiments of the present technology compared to control technology.

Table 1 shows illumination data from multiple sites, wherein some of the sites began using an embodiment of the lighting system in an experimental study.

Table 2 shows a dataset of resident falls from multiple sites, wherein some of the sites began using an embodiment of the lighting system in an experimental study.

Table 3 shows a dataset for resident medication usage from multiple sites, wherein some of the sites began using an embodiment of the lighting system in an experimental study.

Table 4 shows a dataset for resident physical therapy participation from multiple sites, wherein some of the sites began using an embodiment of the lighting system in an experimental study.

Table 5 shows a dataset for resident ambulation performance from multiple sites, wherein some of the sites began using an embodiment of the lighting system in an experimental study.

Table 6 shows a dataset for resident polypharmacy (multiple medications) usage from multiple sites, wherein some of the sites began using an embodiment of the lighting system in an experimental study.

Table 7 shows a dataset for resident demographics from multiple sites, wherein some of the sites began using an embodiment of the lighting system in an experimental study.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In some embodiments, the lighting systems described herein are customizable, programmable, and adaptable. In other embodiments, they are fixed at predetermined settings. The lighting systems provide optimum timing, intensity, and spectrum for one or more or each location within a facility. In some embodiments, such as a medical setting, individual lighting settings are utilized for specific patients or zones containing certain types of patients to provide optimized lighting for a specific category of patient (e.g., based on disease or condition type, age, stage of healing, etc.). In some embodiments, specific times of day and/or locations utilize specific protocols based on historic incidents of fatigue-related problems or injuries for a particular individual or for categories of individuals.

In some embodiments, the optimization of lighting results in one or more positive outcomes, for example, improving and speed of healing, reducing medical errors, increasing staff alertness, reducing falls, reducing sundowners, and reducing the need for psychotropic medication.

In some embodiments, lights are “tunable.” For example, as used herein a “tunable” light source is a light source (e.g., light emitting diode or other light source or lamp) configured to be tuned to one or more alternative parameters (e.g., wavelength, intensity, direction, position, degree of polarization, direction of polarization, etc.). In some embodiments, one or more parameters of a light source are controlled automatically by a controller. In some embodiments, one or more parameters of a light source are controlled manually.

Examples of light source suitable for use in the present disclosure include, but are not limited to, light sources with a plurality of LEDs of different wavelengths that can be turned on and off via a controller, broad spectrum lights (e.g., DC arc lamps) with filters or diffraction gratings to tune the wavelength of light emitted, and the like. In some embodiments, tunable commercial lights sources available from a large number of suppliers (e.g., Acuity Brands (Atlanta, Ga.); Lighting Science (West Warwick, R. I.) and Elite Lighting (Los Angeles, Calif.) are utilized in the present disclosure.

The term “mel-EDI” (alternatively “melanopic EDI” or “m-EDI”), as used herein, refers to the circadian metric that has been adopted by the international commission on lighting (CIE). The CIE issued an international standard, CIE S 026:2018 (CIE 2018), that defines a system for studying the measurement of circadian lighting (CIE, CIE S 026:2018 CIE System for Metrology of Optical Radiation for ipRGC-Influenced Responses to Light. doi: 10.25039/S026.2018. International Standard, available online via http://www.cie.co.at/publications/cie-systemmetrology-optical-radiation-iprgc-influencedresponses-light-0. 2018; CIE, CIE S 026 a-opic Toolbox. doi: 10.25039/S026.2018.TB. Available online at: http://cie.co.at/news/launch-cie-s-026-toolbox-anduser-guide. 2020). Various tools exist to measure parameters and determine mel-EDI (e.g., VISO SYSTEMS). Measurements are based on the position of the eye, in normal application for the task (e.g., eye position in a standing position facing a mirror in the bathroom, or eye position of a person laying down on their back in their bed, etc.). For example, light may be measured with a light meter photosensor positioned in the position of, and directed normal to the eye, as if the photosensor was receiving the same light that the eye would receive in a typical use of the space being measured.

In some embodiments, light sources comprise dimmers to adjust the light level (e.g., measured in lux). The lux is the International System of Units derived unit of illuminance, measuring luminous flux per unit area incident on a surface. In some embodiments, lux values are lux values at the eye position regardless of the individual's orientation and position. In some embodiments, the height is the average height of an individual present in the particular region of the facility receiving the light. In some embodiments, the height is the actual height of the individual, as measured by a sensor or pre-programmed based on a known height and/or position of the individual. In some embodiments, illuminance measurements are based on the incident light on a surface, such as a floor or desk height.

In some embodiments, lighting systems comprise a controller (e.g., comprising a computer processor, computer software, and optionally a user interface such as for example, a computer monitor, a tablet, a smart phone, or smart watch). The controller serves to control all or a portion of the lights in the system. In some embodiments, lights are wired via electrical wires to the system. In other embodiments, lights are controlled wirelessly (e.g., via Bluetooth, near field, WiFi, a combination thereof, or other wireless mechanisms). Various different configurations (e.g., a combination of wired and wireless interfaces) are envisioned by the present disclosure.

In some embodiments, the controller is programmed to automatically adjust lighting based on the region of the facility and/or time of day. In some embodiments, a user manually controls the lighting. In some embodiments, the user interface (e.g., voice, touch, or keypad interface) allows a user to alter the automated protocol. In some embodiments, a plurality of protocols is stored in memory and are selected by a user interface. Such protocols include zone-specific protocols, patient specific protocols (based on patient categories such as health status, age, and the like), room specific protocols, and the like. In some embodiments, the processor is located at a site remote from the facility or on-site. For example, in some embodiments, a service provider manages the lighting systems of two or more different facilities remotely. In some embodiments, patient and staff outcome data is collected from one or more such facilities to allow further optimization based on tracked outcomes (e.g., across a large number of facilities using data pooled from the facilities). In some embodiments, experimental protocols are run to identify improved protocols.

In some embodiments, a facility is divided into zones with different lighting needs. For example, in some embodiments, facilities comprise first, second, and optionally third (or more) zones. In some embodiments, within a zone, lighting is uniform (e.g., all lights of a given type within a zone are set to the same parameter), although each zone may include different types of lighting components that vary from other types of lighting components. For example, in a zone comprising a patient room, all of the bed lights in the zone are set to the same parameters while bed overhead lights in the rooms could be set to the same or different parameters.

The present disclosure is not limited to particular lighting zones. In some embodiments, lighting systems comprise a first zone comprising patient care zones (e.g., patient bedrooms, patient apartments, or common areas). In some embodiments, lighting systems comprise a second zone comprising staff areas (e.g., one or more of nurse's station, hallways, staff rooms, or medical procedure rooms). A facility may have any number of different zones depending on the needs of a given facility (e.g., 1, 2, 3, 4, 5, or more zones per facility).

The present disclosure is not limited to particular facilities. Examples include, but are not limited to, skilled nursing facilities, long term care facilities, hospitals, hospices, assisted living facilities, clinics, correctional facilities (e.g., prisons, jails, youth facilities, etc.) and outpatient surgery centers.

By way of example, the below description provides exemplary zones and lighting protocols illustrated for a skilled nursing home or long-term care facility. The description is for illustrative purposes and does not limit the disclosure.

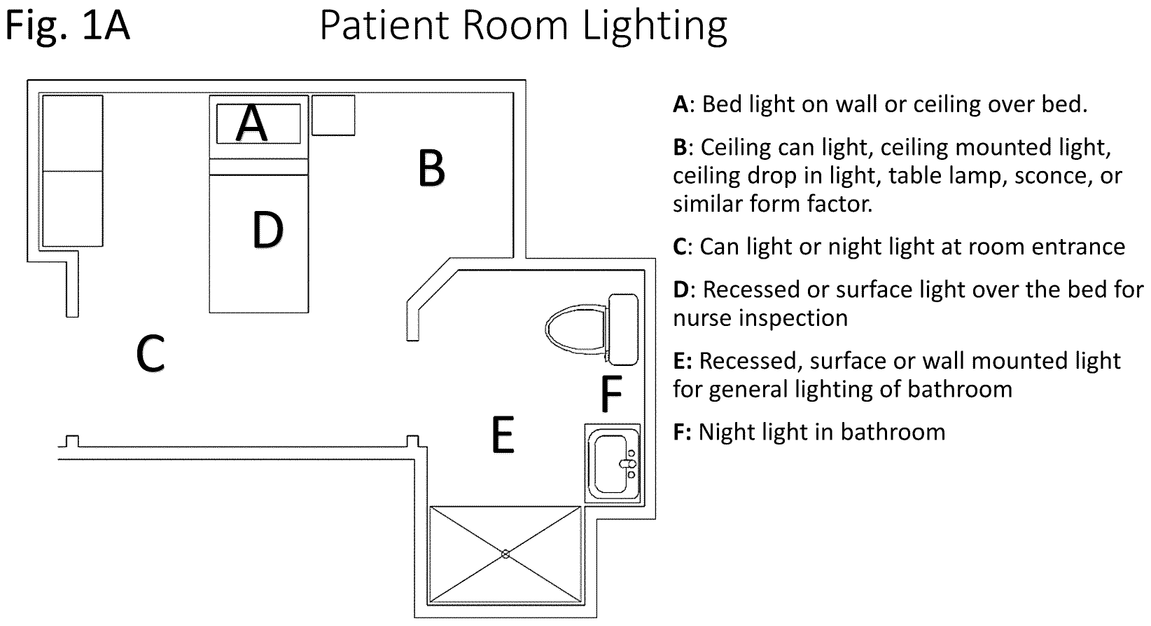

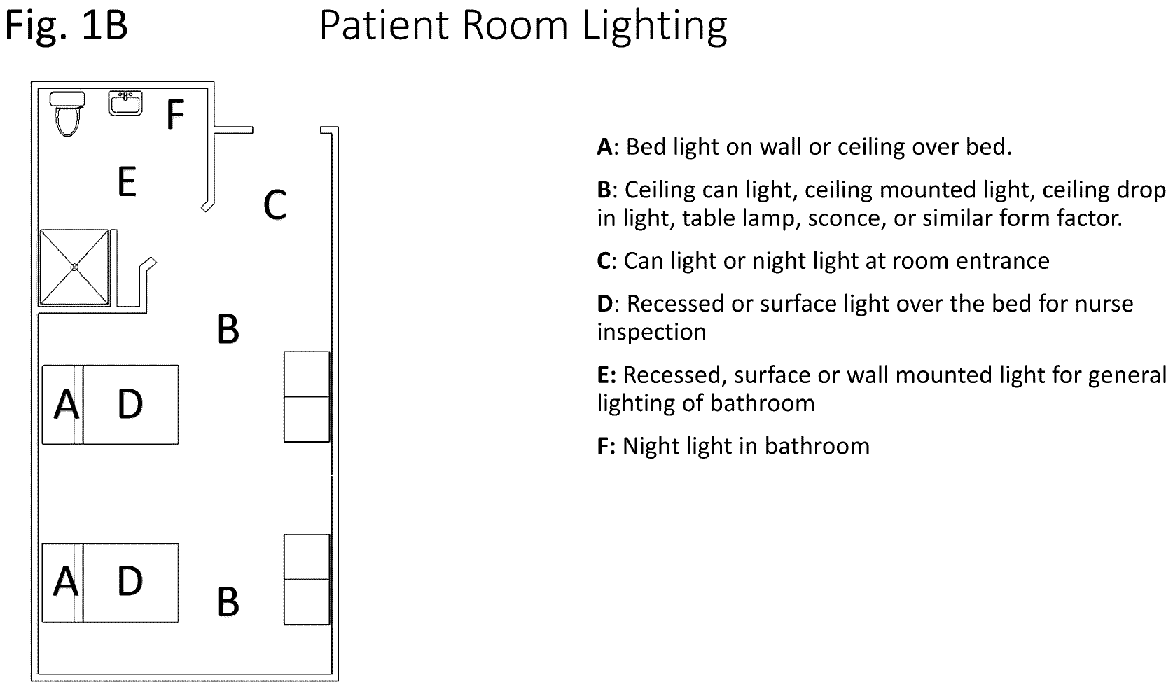

FIGS. 1-4 show exemplary layouts and specifications of certain rooms or areas described below.

FIGS. 1A and 1B shows exemplary layouts of a patient room, which may have beds for one or two patients. The patient room is configured to provide optimal conditions of healing and recovery. The patient room may be located in any type of facility where a subject rests to heal and recover from disease, injury, medical procedures, or the like. Such facilities include, but are not limited to, hospitals, clinics, care homes, nursing homes, mobile medical facilities (e.g., mobile military medical facilities), prisons, ships (e.g., cruise ships), schools, homes, and the like.

Exemplary patient room lighting comprises any or all the following fixture types and placements:
(Highly likely) Fixture A is a wall mounted light fixture mounted at the head of the bed or at the side of the bed, with fixtures having both indirect (lighting aimed up at the ceiling) and direct (light aimed downward) lighting distributions. The indirect and direct components of the lighting distribution are controlled separately for ambient and task lighting, respectively. The downlight component has the ability to dim down to 10 mel-EDI or lower at night, as measured at the eye position when sitting up in the bed.
(Likely) Fixture B is a general light fixture that can be a recessed downlight, recessed area lighting, surface mounted light, wall sconce or portable floor or table lamp, the function of which is to add ambient light and/or additional task lighting.
(Likely) Fixture C is a light fixture that can be a recessed downlight or surface mounted light, or a wall mounted night light, the function of which is to provide a relatively lower level of light at the entrance of the room for nursing staff when inspecting rooms at night.
(Likely) Fixture D is a light fixture that can be a recessed or surface mounted area light located above the bed, supplementing Fixture A for the purpose of providing higher light levels on patient inspection by nurses or doctors when the patient is in their bed.
(Highly likely) Fixture(s) E is a general light fixture that can be a recessed downlight, recessed area lighting, surface mounted light, combination fan/light, or wall mounted light generally mounted over the mirror, or combinations of any of these, the function of which is to provide general lighting in the bathroom.
(Highly likely) Fixture F is a ceiling or wall mounted night light in the bathroom used in lieu of Fixture E when used at night.

Patient room lighting is enhanced throughout the 24-hour day to improve overall patient health and well-being. This results in, for example, reduction of falls, improved healing after illness or medical procedures, and a reduction in medications.

In some embodiments, patient room lights utilize the following protocol: The combination of Fixtures A, B, C, and D provide the following lighting levels as measured at the eye in the normal sitting position in the bed:
7:00 AM-10:00 AM: 40 mel-EDI minimum
10:00 AM-6:00 PM: 100 mel-EDI minimum
6:00 PM-8:00 PM: 40 mel-EDI maximum
8:00 PM-10:00 PM: 20 mel-EDI maximum
10:00 PM-7:00 AM: OFF, night light only.

The resident may turn on Fixture A, however the light level setting from the system allows it to go no higher than 10 mel-EDI if turned on by the resident. Medical staff may adjust lighting to reach 100 mel-EDI minimum at any time.

Fixtures E and F: Fixture E provides sufficient lighting for daytime use of the bathroom and is used for short durations only; thus lighting should meet applicable lighting standards. Fixture F should provide lower than 10 mel-EDI at the eye from the standing position facing the sink.

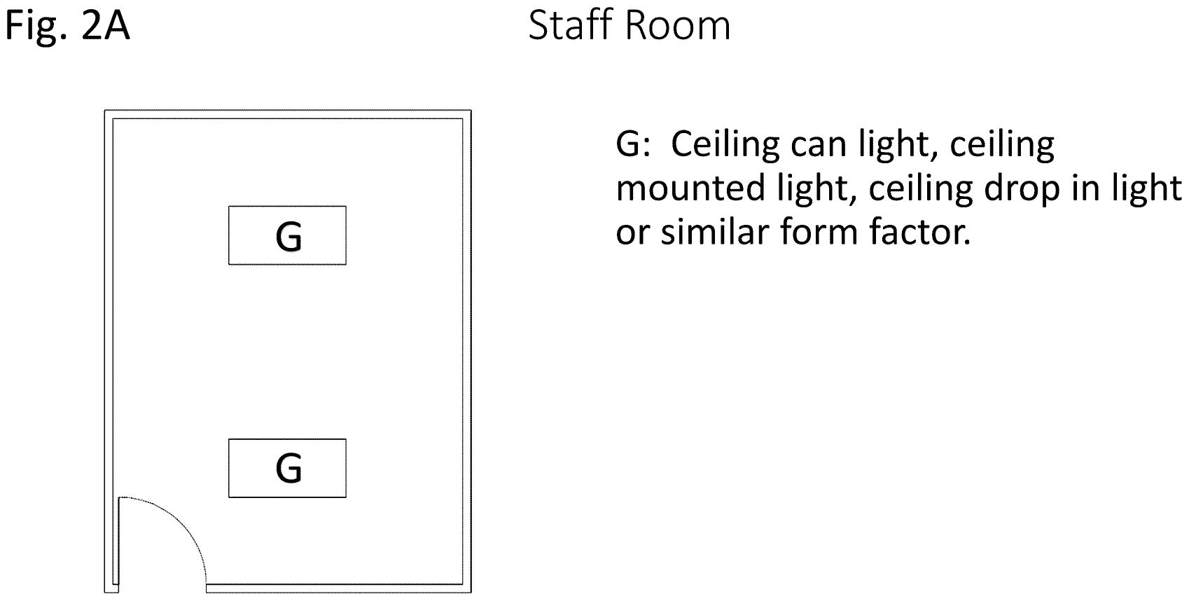

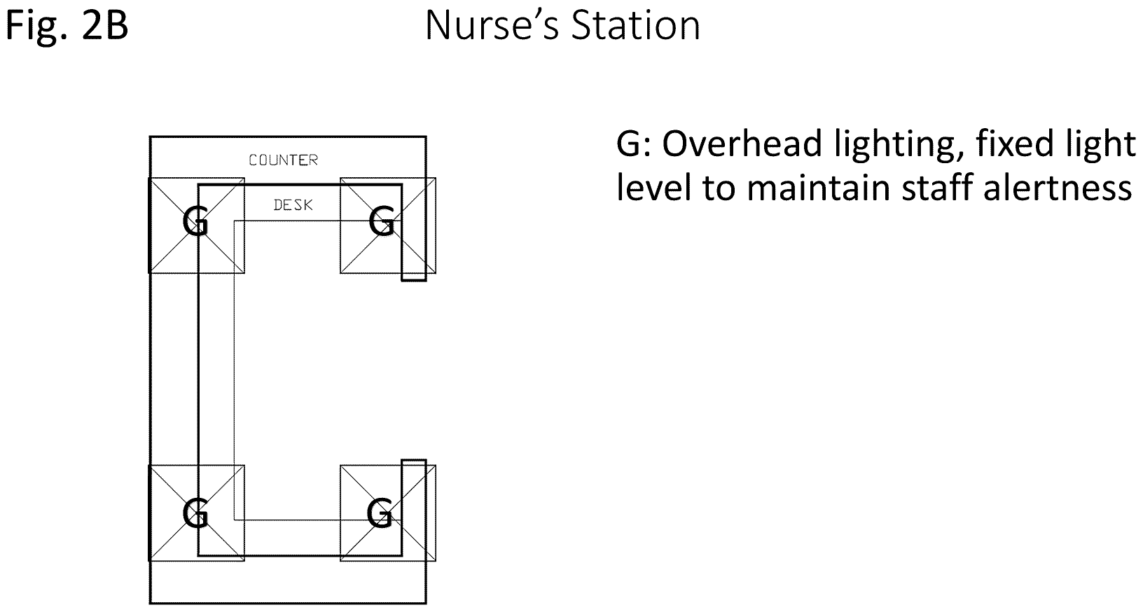

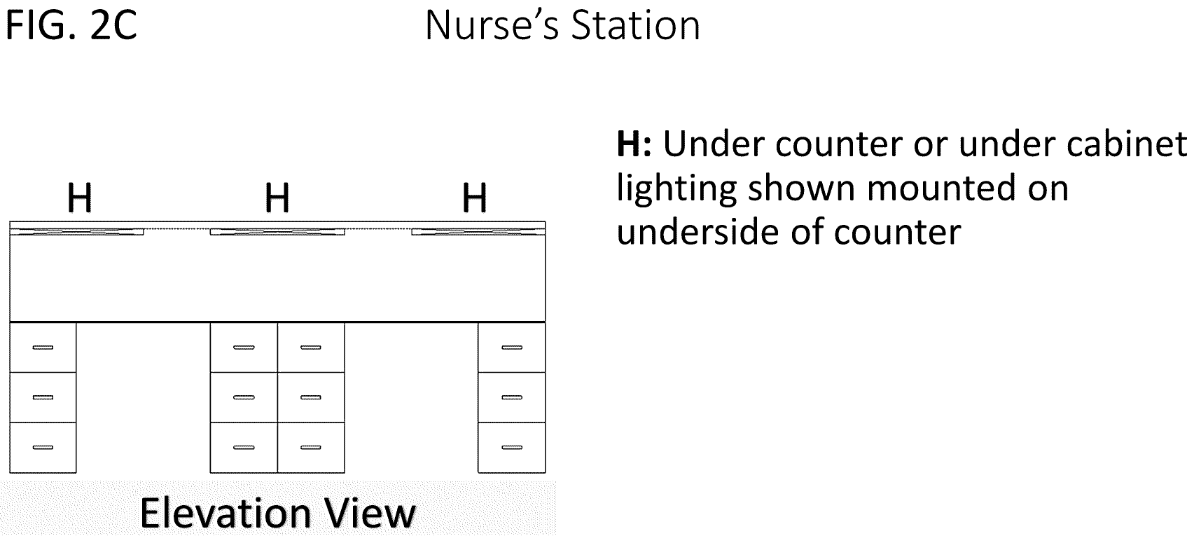

FIG. 2A shows an exemplary layout of a staff area. FIG. 2B and FIG. 2C show exemplary layouts of a nursing station. Staff areas and nursing stations may be an open area within a common area or a separate room that is configured to provide optimal conditions of maintaining staff attentiveness. The staff area/room may be located in any type of facility where one or more staff members work, recreate, dine, or otherwise congregate. Such facilities include, but are not limited to, hospitals, clinics, care homes, nursing homes, mobile medical facilities (e.g., mobile military medical facilities), prisons, ships (e.g., cruise ships), schools, homes, and the like. The staff area/room may be a nurse's station, a meeting room (e.g., conference room), break room, dining room, office, training room, lecture hall, and the like.

Exemplary staff area lighting comprises any or all the following fixture types and placements:
(Highly Likely) Fixture G is a light fixture that can be a recessed or surface mounted area source for generally lighting the staff area/room.
(Highly Likely) Fixture H is an undercounter or under cabinet linear light fixture that focuses the lighting on the task area (work desk or work counter).

Staff room lighting is enhanced throughout the 24-hour day to improve overall staff performance. This results in, for example, reduction of errors and increased job satisfaction.

In some embodiments, staff room lights utilize the following protocol:

Light levels are constant throughout a 24-hour period to maintain alertness of staff, with lighting within rooms allowed to be controlled by occupancy sensors, but not dimmers. The combination of lighting fixtures within staff areas generates a minimum of 250 mel-EDI, 24 hours a day, as measured vertically at the eye in a sitting position (48″ AFF) within the room. The combination of lighting fixtures within nurse's stations generate a minimum 150 mel-EDI 24 hours a day, as measured vertically at the eye in a sitting position (48″ AFF) at the nurse's station desk.

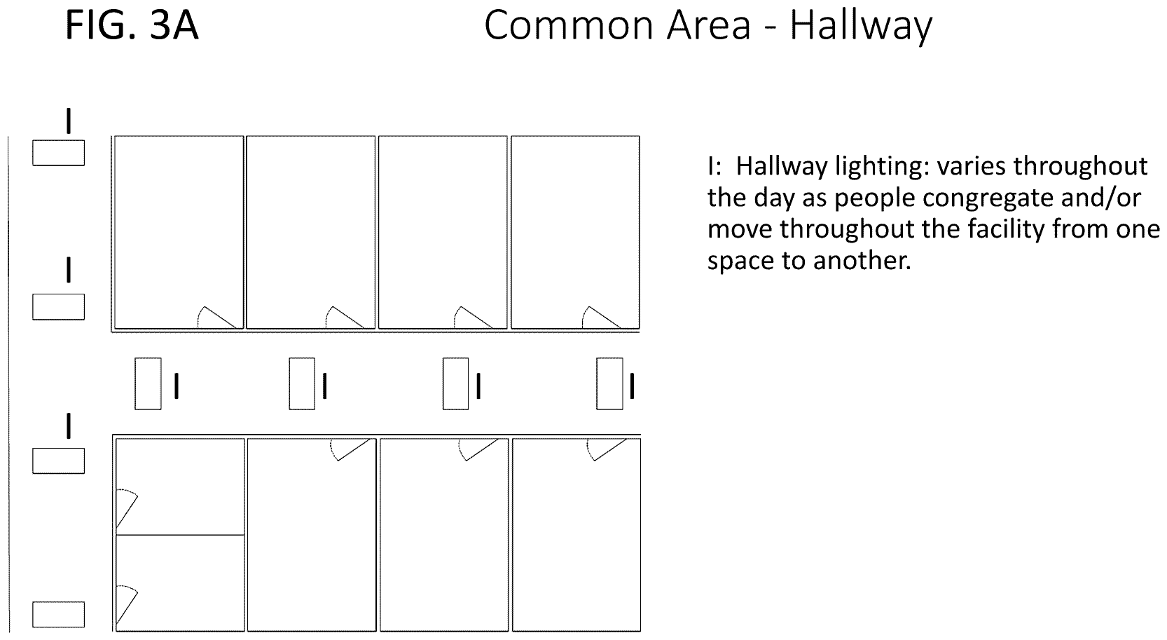

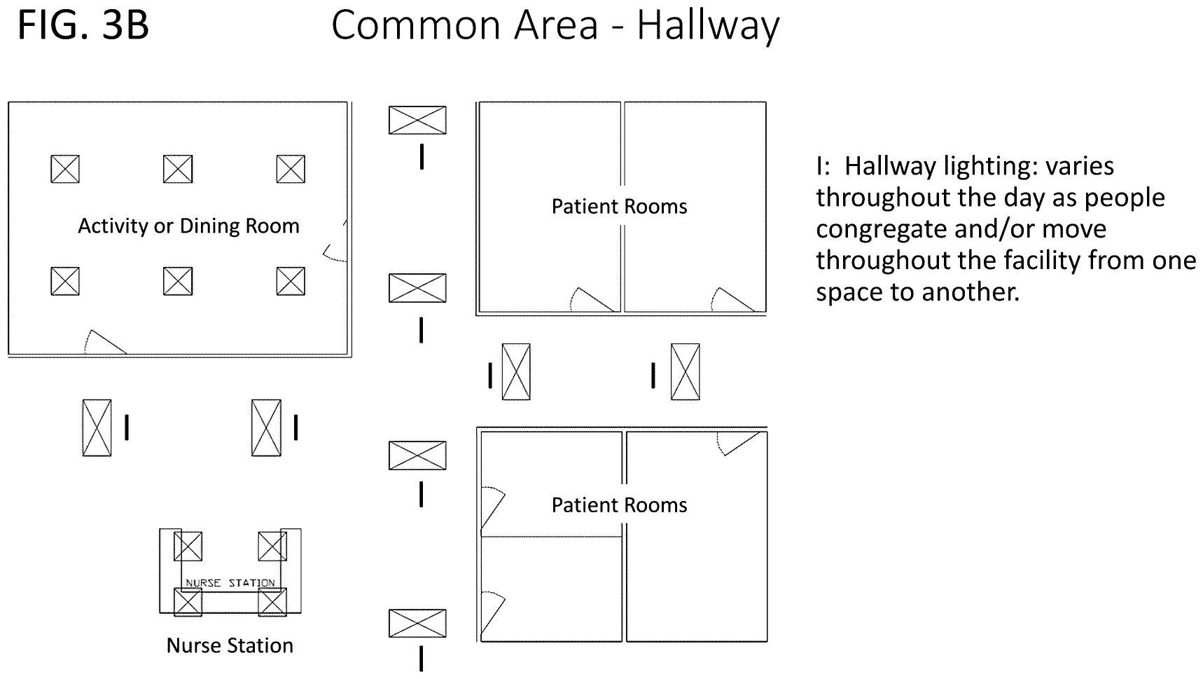

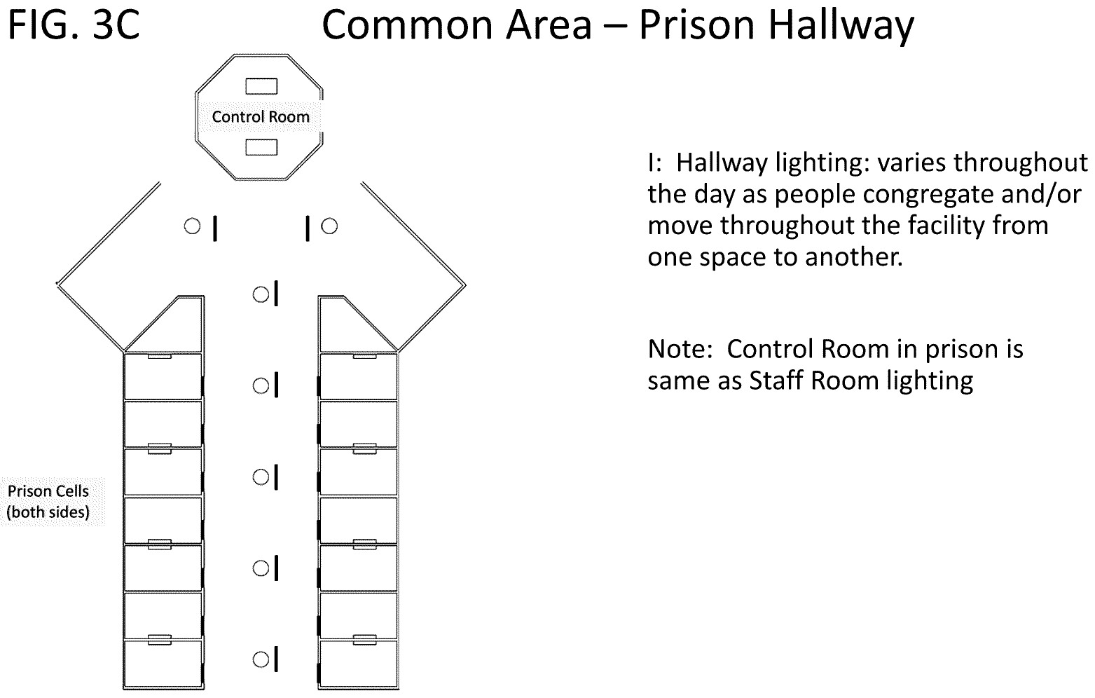

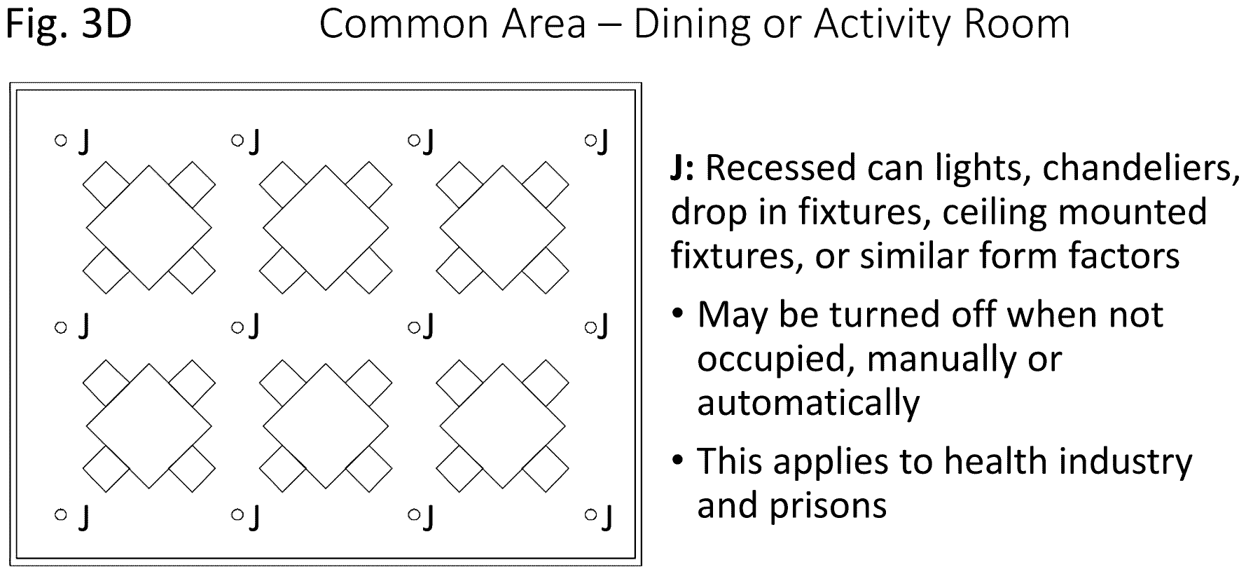

FIG. 3A, FIG. 3B and FIG. 3C show exemplary layouts of common areas in the context of other room types. Common areas are configured to provide optimal conditions for mixed use by a variety of individuals or groups as well as transition areas between room types. Common areas may be located in any type of facility where a variety of different people visit. Such facilities include, but are not limited to, hospitals, clinics, care homes, nursing homes, mobile medical facilities (e.g., mobile military medical facilities), prisons, ships (e.g., cruise ships), schools, homes, and the like. The common area may be a hallway, lobby, reception area, meeting room, cafeteria, dining room, recreational areas, and the like.

Exemplary staff area lighting comprise any or all the following fixture types and placements:
(Highly Likely) In FIG. 3A, FIG. 3B, and FIG. 3C, fixture I is a recessed or surface mounted hallway light fixture that changes light level and spectrum during the 24-hour period to increase alertness and wakefulness during the day, and to minimize sleep disruption for the inhabitants of the adjacent sleeping quarters.
(Highly Likely) In FIG. 3D Fixture J is a recessed can or area light, surface area light, chandelier, or similar form factor providing general lighting to the space; there are commonly mixed types of luminaires in these spaces.

Common area lighting is enhanced throughout the 24-hour day to balance the mixed use and the needs of a variety of different individuals or groups that have shared or divergent needs (e.g., a patient preparing for evening rest, a staff person on an extended hour shift, a visitor, etc.).

In some embodiments, common area lights utilize the following protocol:
Hallways, lobbies, waiting rooms adjacent to sleeping quarters:
7:00 AM-10:00 AM: 100 mel-EDI minimum
10:00 AM-4:00 PM: 135 mel-EDI minimum
4:00 PM-6:00 PM: 100 mel-EDI minimum
6:00 PM-8:00 PM: 60 mel-EDI minimum, 100 mel-EDI maximum
8:00 PM-10:00 PM: 20 mel-EDI minimum, 60 mel-EDI maximum
10:00 PM-7:00 AM: 20 mel-EDI maximum.

Lighting in corridors are on 24 hours per day with this protocol and should have emergency lighting override ability to meet local codes in case of fire or emergency.

The protocol for Dining and Activity Rooms is as follows:

Light levels shall be constant when occupied to maintain alertness. The combination of lighting fixtures within Dining Rooms will generate a minimum of 135 mel-EDI when occupied, as measured vertically at the eye in a sitting position (48″ AFF) within the room. The combination of lighting fixtures within Activity Rooms will generate a minimum 150 mel-EDI when occupied, as measured vertically at the eye in a sitting position (48″ AFF) within the room. For either room, when unoccupied, the lighting can be off.

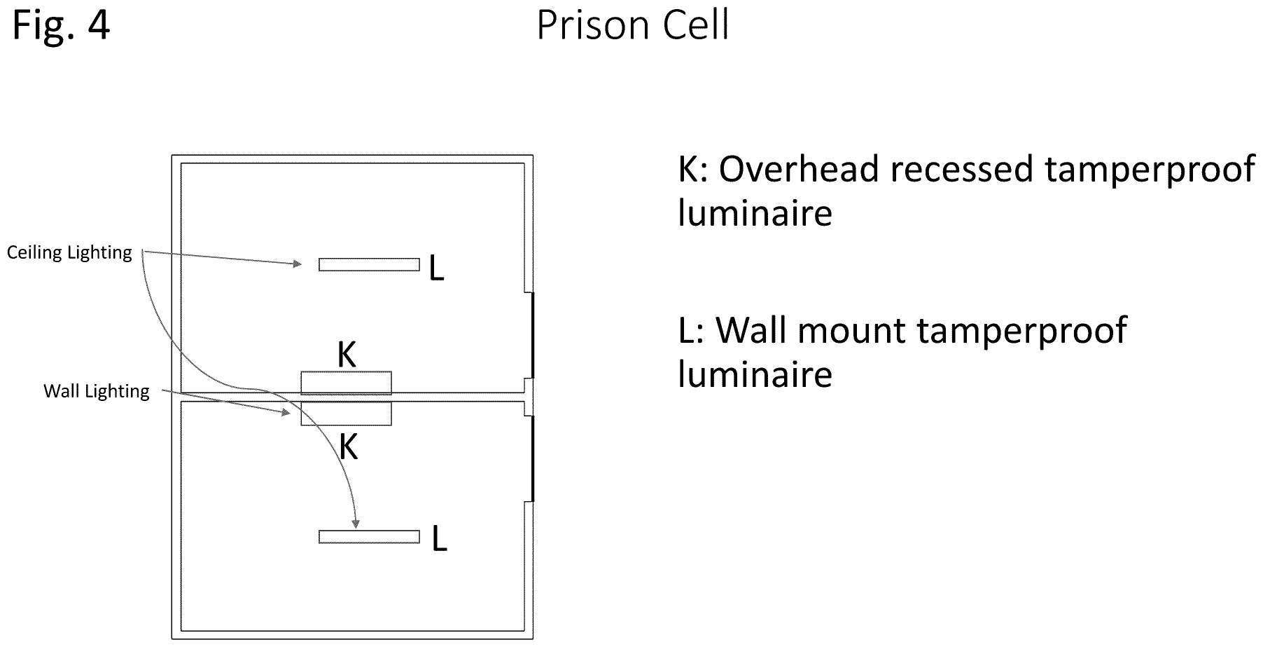

FIG. 4 shows an exemplary layout of a prison cell. The prison cell is configured to provide optimal conditions of a prisoner's physical and mental health and well-being. The prison cell may be located in any type of facility where a subject is confined for one or more days. Such facilities include, but are not limited to, prisons, jails, hospitals, mental health facilities, ships, schools, homes, and the like.

Exemplary prison cell lighting comprises any or all the following fixture types and placements:
(Highly likely) Fixture K is a tamperproof wall mounted light fixture.
(Highly Likely) Fixture L is a tamperproof recessed general light fixture.

Prison room lighting is enhanced throughout the 24-hour day to improve overall physical and mental health and well-being of individuals restrained therein. This results in, for example, improved mental and physical health, reduction in disciplinary action, and increase resident and staff safety.

In some embodiments, prison room lights utilize the following protocol:

The combination of Fixtures K and L provide the following lighting levels as measured at the eye in the normal sitting position in the bed:
7:00 AM-10:00 AM: 40 mel-EDI minimum
10:00 AM-6:00 PM: 100 mel-EDI minimum
6:00 PM-8:00 PM: 40 mel-EDI maximum
8:00 PM-10:00 PM: 20 mel-EDI maximum
10:00 PM-7:00 AM: OFF, night light only.

A summary of exemplary lighting conditions, as measured in mel-EDI at the eye, and in the eye position (horizontal or vertical) for the average use case within the Area Type provided in Table 8 below.

| | | Start time: | | | | | |
|---|---|---|---|---|---|---|---|
| | | 7 AM | 10 AM | 4 PM | 6 PM | 8 PM | 10 PM |
| | | End time: | | | | | |
| | | 10 AM | 4 PM | 6 PM | 8 PM | 10 PM | 7 AM |
| | | Time period: | | | | | |
| | | Early | Mid- | | Early | | |
| | | morning | morning | Afternoon | evening | Evening | Bedtime |
| Area type | | | | | | | |
| Sleeping quarters | Min or max: | Min | Min | Min | Max | Max | Max |
| Patient | | 40 | 100 | 100 | 40 | 20 | Night light only |
| room/prison cell | | | | | | | Resident switch to max 10 |
| | | | | | | | Medical staff switch to 100 |
| Common areas | Min or Max: | Min | Min | Min | Min | Min | Max |
| Hallway/circulation | | 100 | 135 | 100 | 60 | 30 | 10 |

| Activity areas | Min or Max: | Min |
|---|---|---|
| Activity area | 150 when occupied, otherwise OFF | |
| Dining room | 135 when occupied, otherwise OFF | |
| Staff areas | Min or Max: | Min |

| | |
|---|---|
| Nursing | 150, 24 hours per day |
| station/control room | |
| Staff area | 250, occupancy sensor controlled |
| (break room) | |

Experiments were conducted during the development of the technology whereby embodiments of the above systems were employed in nursing home facilities. Using the described protocol, the following outcomes were observed: A reduction in the number of falls by 43%, a reduction in the number of sundowners by 38%, a reduction in the need for psychotropic meds by 10%, a reduction in harmful medical errors by 33%, and a reduction in energy consumption by 65%.

In some embodiments, the lighting system is automated for a controlled environment. In some embodiments, the lighting system is tailored for environments where there is a need for individuals to be alert during the day and sleeping at night. In some embodiments, the lighting system is tailored for environments where there is a need for individuals to be alert during the day and sleeping at night, and some individuals need to be alert in the night as well. For example, in some environments, residents as well as daytime staff will be alert during the day, but will be sleeping at night; however, the night shift staff will need to be alert during the night. These example environments include, but are not limited to, hospitality (hotels, motels, etc.), airplane cabins, assisted living facilities, residential lighting (homes, apartment buildings, etc.), military facilities, prisons, and dormitories or other controlled residential facilities.

The lighting system benefits individual's health by providing proper circadian lighting. These benefits include, but are not limited to, reduced sundowning symptoms (up to 50% lower), improved sleep quality, reduced need for psychotropic medications (up to 20% less), reduced anxiety, increased activity level during the day, improved mood, reduced falls, improved cognition, improved recovery time, quicker reaction time, alleviated seasonal affective disorder, and adjusted circadian rhythm for night shift workers (which reduced errors during night shifts due to increased worker alertness). Further, the lighting system reduces energy consumption for lighting.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

### Examples

One embodiment of the lighting system for use in a facility (“the lighting system”) was utilized in a prospective experimental study of two pairs of care homes. Each care home had standardized care and protocols. One pair (the “experimental sites”) was implemented with the lighting system and the other pair (the “control sites”) continued using normal lighting. The study was conducted over a two-year period, with the experimental sites being implemented with the lighting system after 12 months.

The experimental sites had the lighting system set to a dynamic lighting schedule (DLS), where the lighting of the site would change throughout the day and night to modulate the intensity and spectrum of ambient lighting which alters the melanopic illuminance (a measure of the strength of the stimulus of the principal photoreception that primarily mediates the nonvisual effects of light) experienced by the care home resident.

Before the lighting system was implemented, the experimental sites used fluorescent lamps with a Correlated Color Temperature (CCT) ranging from 2700K to 3500K. The control sites also used fluorescent lamps, mixed between 2700K and 6500K or 2700K and 4000K respectively.

The lighting system implementation was as follows: common areas (e.g., dining rooms and sitting rooms) and hallways had the DLS set to (1) a graduated day setting with high-intensity, blue-enriched white light from 6 am to 6 pm (60% max intensity from 6 am-10 am, 100% max intensity from 10 am-3 pm, 60% max intensity from 3 pm-6 pm), (2) an evening setting with low-intensity, blue-depleted white light from 6 pm to 10 pm (60% max intensity), and (3) a night setting with lower-intensity, blue-depleted white light from 10 pm to 6 am; in resident bedrooms, the DLS was set to a high-intensity, blue enriched white light from 6 am to 6 pm; and in the activity room, there was blue-enriched, solid-state white light (5000K) installed, for when residents attending evening activities between 7 pm to 9 pm. The lighting system DLS was mostly automated, but simple instructions were given to the care home staff to make the appropriate lighting adjustments.

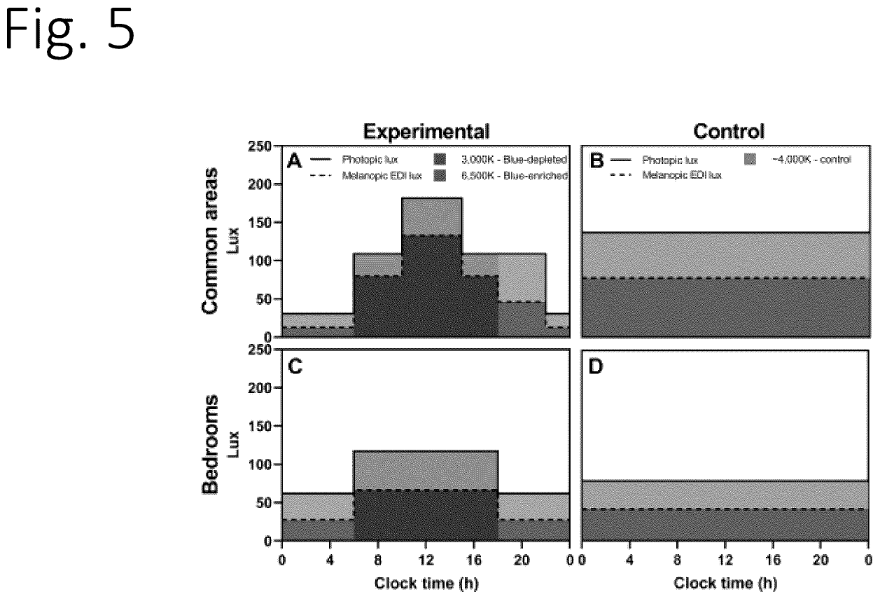

Illumination measurements were made using a ColorMunki colorimeter and were converted to International Commission on Illumination (CIE) a-opic equivalent daylight (D65) illuminance (EDI) units (the SI units for quantifying photic stimuli for nonvisual responses). The illumination measurements were taken for both the 100% max intensity day and the low-intensity night lighting. Table 1 records the illumination measurements, specifically the radiometric, photometric, and spectral characteristics of the lighting system for both the experimental and control sites. FIG. 5 illustrates the illumination measurements in the common areas and the bedrooms set to the DLS of the lighting system throughout the day compared to the control lighting. The 60% max intensity day and evening lighting measurements were estimated based on the measured 100% max intensity day and the low-intensity night lighting values.

The measured outcome for the study was the rate of falls per 1000 resident-days. “Falls” was defined per the Center for Medicare and Medicaid Services definition: “unintentionally coming to rest on the ground, floor, or other lower level but not as a result of an overwhelming external force.” The number of falls was determined by retrospective review of medical records, which were documented by the care providers at each site. The data for number of falls was compiled into a data set, the Minimum Data Set (MDS) records. The MDS record was available for 562 of the 758 (74%) of residents who contributed to the falls dataset. In the MDS records reporting at least one fall, data was dichotomized into either “no injury” or “injurious”. No injury was defined as a frequency of 1 or 2+ falls exclusively in the no-injury category. Injurious was defined as a frequency of 1 or 2+ falls in the injury and major injury categories.

Resident demographics, including age, sex, dementia, and resident-days (at the site) were recorded in the MDS pre-lighting upgrade and post-lighting upgrade (see Table 7). The resident demographics data allowed for multiple models of the data to be analyzed.

Further, the MDS included medication, ambulation, transfer, and physical therapy as variables. Medication usage was assessed based on the number of days a resident received one or more of 8 pharmacological classifications of medicine over the last seven days: antipsychotic, antianxiety, antidepressant, hypnotic, anticoagulant, antibiotic, diuretic, and opioid. Receiving a medication on at least one of the 7 days counted as using that medication. Within medications is the variable “fall-related medications”, defined as receiving one or more medications associated with increased fall risk”-antipsychotic, antianxiety, antidepressant, hypnotic, diuretic, and opioid, and “polypharmacy”, defined as receiving 0, 1, 2, or 3+ medications. The medications, fall-related medications, and polypharmacy data was calculated from the MDS-recorded medication usage (see Table 3 and Table 6).

A resident having received physical therapy was determined by the MDS record. A resident receiving physical therapy was defined as having more than 15 minutes of physical therapy on at least one of the last seven days (see Table 4).

Ambulation and transfer were assessed by the MDS record. “Resident's performance” includes how well the resident moved between locations in his/her room and adjacent corridor on the same floor (locomotion on the unit) and how the resident moved between surfaces including to or from: bed, chair, wheelchair, standing position (transfer). Performance for locomotion on the unit and transfer were split into two statuses: independent/supervision only or requiring assistance/dependent (see Table 5).

The data accounted for resident demographics, and MDS record medication, physical therapy, ambulation, and transfer between lighting conditions, stratified by observation interval (pre/post lighting upgrade), using Wilcoxon and Fisher's Exact tests (see Tables 3-7). The rate ratios (RRs) for falls were estimated using generalized linear mixed models (GLMM), accounting for randomness and error. Odds ratios for injurious falls were also estimated using GLMM. Exploratory analysis of the data was also done using GLMM-specifically the distribution in the average number of falls per resident-which was conducted between daytime (e.g., 6 am-6 pm) and nighttime (e.g., 6 pm-6 am), as well as between the intervals pre/post-lighting upgrade at both the experimental and control sites.

The results are as follows. Throughout the entire two year study, across all the sites, there was a total of 834 falls. Pre-lighting upgrade, the rate of falls was the same across the experimental and control sites (using data recorded from 515 residents), with a fall rate of 6.94 vs. 6.62 falls per 1000 resident-days, respectively (see Table 2). Post-lighting upgrade, the rate of falls was 43% lower at the experimental sites compared to the control sites (using data recorded from 438 residents), with a fall rate of 4.82 vs. 8.44 falls per 1000 resident-days, respectively (see Table 2). Further, the data suggests that post-lighting upgrade there was a decrease in the number of injurious falls at the experimental sites compared to the control sites.

In another model, when adjusting for age, sex, and proportion of residents with dementia, the rate of falls remained significantly lower at the experimental sites compared to the control sites, with 5.34 vs. 9.22 falls per 1000 resident-days, respectively. In yet another model, when adjusting for age, sex, dementia, physical therapy, ambulation transfer, and medication, the rate of falls remained lower at the experimental sites compared to the control sites, with 4.06 vs. 6.61 falls per 1000 resident-days, respectively.

Further, the data showed that the distribution of falls by time of day (daytime vs. nighttime) was significantly different, post-lighting upgrade, between the experimental and control sites (using data from 247 residents who had a fall). Post-lighting upgrade, at the control sites, 58% of the falls occurred during nighttime, while at the experimental sites, 55% of the falls occurred during daytime. There was no difference in the distribution of falls between the experimental and control sites pre-lighting upgrade.

Finally, regarding all the different embodiments of the lighting system, various features and advantages are set forth in the following claims.

TABLE 1

| | | | | Radiometric and | | | | | | | |
|---|---|---|---|---|---|---|---|---|---|---|---|
| | | | | Photometric Values | | | | | | | |
| | | | | (380-750 mm inclusive) | | | | | | | |
| | | | | | Photopic | Retinal Photopigment Weighted | | | | | |
| Lighting Condition | | | | Irradiance | illuminance | Illuminances (α-epic EDI lux) | | | | | DER |
| Condition | Setting | Location | CCT | μW/cm³ | lux | S Cone | Melanopsin | Rod | M Cone | L Cone | Melanopsin |
| Experimental | Day | Common | 4712 ± | 59.0 ± | 181.5 ± | 131.4 ± | 132.8 ± | 141.5 ± | 166.8 ± | 180.7 ± | 0.73 |
| | | areas | 863 | 39.8 | 107.6 | 94.0 | 91.6 | 94.4 | 102.9 | 107.0 | |
| | | Bedroom | 3440 ± | 38.7 ± | 117.2 ± | 37.9 ± | 66.1 ± | 74.3 ± | 99.5 ± | 117.8 ± | 0.58 |
| | | | 552 | 18.9 | 59.0 | 38.2 | 38.5 | 41.6 | 51.9 | 59.0 | |
| | Night/ | Common | 2694 ± | 9.8 ± | 30.7 ± | 9.5 ± | 12.9 ± | 15.4 ± | 24.0 ± | 31.3 ± | 0.42 |
| | Evening | areas | 670 | 16.3 | 54.3 | 16.0 | 22.5 | 27.1 | 42.6 | 55.5 | |
| | | Bedroom | 2768 ± | 21.4 ± | 62.0 ± | 18.2 ± | 27.2 ± | 32.5 ± | 49.1 ± | 63.0 ± | 0.44 |
| | | | 154 | 11.8 | 33.6 | 11.2 | 15.9 | 18.7 | 27.2 | 33.9 | |
| Control | — | Common | 3842 ± | 38.8 ± | 136.6 ± | 68.6 ± | 77.1 ± | 88.9 ± | 119.9 ± | 135.0 ± | 0.56 |
| | | areas | 715 | 30.2 | 106.5 | 63.5 | 86.2 | 74.4 | 95.6 | 104.8 | |
| | — | Bedroom | 3593 ± | 21.8 ± | 77.0 ± | 33.0 ± | 39.9 ± | 46.9 ± | 66.0 ± | 76.4 ± | 0.52 |
| | | | 445 | 21.3 | 75.2 | 34.9 | 42.5 | 48.6 | 65.8 | 74.6 | |

DER, daylight (D65) efficacy ratio.
Values represent mean ± SD.

TABLE 2
Total Falls, Patient Days and Fall Rate for Control and
Experimental Sites Before and After the Lighting Upgrade

| | | Number | Fall Rate/1000 |
|---|---|---|---|
| | Number | of Patient | Patient Days |
| | of Falls | Days | [95% Cl] |
| Pre lighting upgrade | | | |
| Control sites | 190 | 28,305 | 6.6 [5.6-7.9] |
| Experimental sites | 297 | 44,005 | 6.9 [4.8-10.1] |
| Post lighting upgrade | | | |
| Control sites | 174 | 19,903 | 8.4 [6.6-10.1] |
| Experimental sites | 173 | 34,266 | 4.8 [3.6-6.4] |

Fall rates are reported for the unadjusted models.

TABLE 3
The Number and Percentage of Residents With MDS-Recorded Medication Usage
Control and Experimental Sites Before and After the Lighting Upgrade

| | Pre Lighting Upgrade | | | | | | | | |
|---|---|---|---|---|---|---|---|---|---|
| | | | | | Experimental | | | | Fisher's |
| Pharmacological | Control (n = 133) | | | | (n = 257) | | | | Exact |
| Classification | No | | Yes | | No | | Yes | | P Value |
| Antipsychotic | 117 | (88%) | 16 | (12%) | 218 | (85%) | 39 | (15%) | 0.45 |
| Antianxiety | 109 | (82%) | 24 | (18%) | 204 | (79%) | 53 | (21%) | 0.59 |
| Antidepressant | 86 | (65) | 47 | (35) | 118 | (46) | 139 | (54) | <.001 |
| Hypnotic | 131 | (98) | 2 | (2) | 255 | (99) | 2 | (1) | .61 |
| Anticoagulant | 93 | (70) | 40 | (30) | 180 | (70) | 77 | (30) | 1.00 |
| Antibiotic | 87 | (65) | 46 | (35) | 173 | (67) | 84 | (33) | .73 |
| Diuretic | 64 | (48) | 69 | (52) | 141 | (55) | 116 | (45) | .24 |
| Opioid [symbol] | 86 | (65) | 47 | (35) | 144 | (57) | 108 | (43) | .16 |
| Fall-related | 23 | (17) | 110 | (83) | 36 | (14) | 221 | (86) | .46 |
| medications [symbol] | | | | | | | | | |

| | Post Lighting Upgrade | | | | | | | | |
|---|---|---|---|---|---|---|---|---|---|
| | | | | | Experimental | | | | Fisher's |
| Pharmacological | Control (n = 116) | | | | (n = 219) | | | | Exact |
| Classification | No | | Yes | | No | | Yes | | P Value |
| Antipsychotic | 105 | (91%) | 11 | (9%) | 186 | (85%) | 33 | (15%) | .18 |
| Antianxiety | 96 | (83%) | 20 | (17%) | 181 | (82%) | 38 | (17%) | 1.00 |
| Antidepressant | 67 | (58) | 49 | (42) | 108 | (49) | 111 | (51) | .17 |
| Hypnotic | 116 | (100) | 0 | (0) | 218 | (99.5) | 1 | (0.5) | 1.00 |
| Anticoagulant | 80 | (69) | 36 | (31) | 156 | (71) | 63 | (29) | .71 |
| Antibiotic | 78 | (67) | 38 | (33) | 150 | (68) | 69 | (32) | .81 |
| Diuretic | 57 | (49) | 59 | (51) | 118 | (53) | 103 | (47) | .57 |
| Opioid [symbol] | 73 | (63) | 43 | (37) | 118 | (54) | 101 | (46) | .13 |
| Fall-related | 19 | (16) | 97 | (84) | 29 | (13) | 190 | (87) | .51 |
| medications [symbol] | | | | | | | | | |

[symbol]

Data on opioid use missing for n = 5 residents at experimental sites pre lighting upgrade. [symbol]
Indicates residents receiving at least 1 medication associated with increases risk of falls (antipsychotic, antianxiety, antidepressant, hypnotic, diuretic, opioid). [symbol]
indicates data missing or illegible when filed

TABLE 4
The Number and Percentage of Residents With and Without MDS-Recorded Physical
Therapy at Control and Experimental Sites Before and After the Lighting Upgrade

| | Pre Lighting Upgrade | | | | | Post Lighting Upgrade | | | | |
|---|---|---|---|---|---|---|---|---|---|---|
| | | | Experimental | | Fisher's | | | Experimental | | Fisher's |
| | Control (n = 133) | | (n = 257) | | Exact | Control (n = 116) | | (n = 219) | | Exact |
| Physical Therapy | No | Yes | No | Yes | P Value | No | Yes | No | Yes | P Value |
| Received at least 15 | 23 (17) | 110 (83) | 59 (23) | 198 (77) | .2380 | 28 (22) | 90 (78) | 85 (39) | 134 (61) | .0023 |
| min of physical | | | | | | | | | | |
| therapy on at least 1 | | | | | | | | | | |
| day in the past week | | | | | | | | | | |

TABLE 5
The Number and Percentage of Residents With and Without MDS-Recorded Locomotion and Transfer
Impairment at Control and Experimental Sites Before and After the Lighting Upgrade

| | Pre Lighting Upgrade | | | | | Post Lighting Upgrade | | | | |
|---|---|---|---|---|---|---|---|---|---|---|
| | Control | | Experimental | | | Control | | Experimental | | |
| | (n [symbol] = 128; | | (n [symbol] = 247; | | Fisher's | (n [symbol] = 114; | | (n [symbol] = 215; | | Fisher's |
| Ambulation/ | n [symbol] = 132) | | n [symbol] = 253) | | exact | n [symbol] = 115) | | n [symbol] = 216) | | Exact |
| Transfer | Indp. | Assist | Indp. | Assist. | P Value | Indp. | Assist. | Indp. | Assist. | P Value |
| Locomotion on | 15 (12) | 113 (88) | 68 (28) | 179 (72) | .0004 | 22 (81) | 92 (81) | 41 (19) | 174 (81) | 1.000 |
| unit Transfer | 9 (7) | 123 (93) | 39 (15) | 215 (85) | .0151 | 11 (10) | 104 (90) | 19 (9) | 197 (91) | .8420 |

Assist. = Assistance; Indp. = independent; N [symbol] = number of participants in locomotion analysis; N [symbol] = number of participants in transfer analysis. [symbol]
indicates data missing or illegible when filed

TABLE 6
The Number and Percentage of Residents With Polypharmacy at Control
and Experimental Sites Before and After the Lighting Upgrade

| | Pre Lighting Upgrade | | Post Lighting Upgrade | |
|---|---|---|---|---|
| No. of | Control | Experimental | Control | Experimental |
| Medications | (n = 133) | (n = 252) [symbol] | (n = [symbol] 6) | (n = 219) |
| 0 | 23 (17) | 35 (14) | 19 (16) | 29 (13) |
| 1 | 42 (32) | 72 (29) | 38 (33) | 72 (33) |
| 2 | 50 (38) | 83 (33) | 38 (33) | 64 (29) |
| 3 [symbol] | 18 (14) | [symbol] (25) | 21 (18) | 54 (25) |
| [symbol] value | .0852 | | .5149 | |

[symbol]

Data missing for n = 5 residents at experimental sites pre lighting upgrade. [symbol]
indicates data missing or illegible when filed

TABLE 7
Demographic Information for Control and Experimental
Sites Before and After the Lighting Upgrade

| Demographic | Control Sites | Experimental Sites | P |
|---|---|---|---|
| Pre lighting upgrade | | | |
| n | 224 | 291 | — |
| Age, mean ± SD y | 83.5 ± 10.2 | 82.1 ± 11.4 | .24 [symbol] |
| Females, n (%) | 119 (53.1) | 177 (60.8) | .09 [symbol] |
| Dementia, n (%) [symbol] | 64 (29.4) | 92 (33.3) | .38 [symbol] |
| Resident-days, | 126.4 ± 150.8 | 151.2 ± 152.6 | .02 [symbol] |
| mean ± SD days | | | |
| Post lighting upgrade | | | |
| n | 203 | 235 | — |
| Age, mean ± SD y | 80.4 ± 12.2 | 80.6 ± 11.9 | .98 [symbol] |
| Females, n (%) | 119 (58.6) | 147 (62.6) | .43 [symbol] |
| Dementia, n (%) [symbol] | 57 (29.5) | 94 (41.6) | .01 [symbol] |
| Resident-days, | 98.0 ± 110.8 | 145.8 ± 128.7 | .0003 [symbol] |
| mean ± SD days | | | |

[symbol]

Wilcoxon 2-sample test. [symbol]
Fisher's Exact test. [symbol]
Dementia status was unknown in a subset of the patients (Control: n = 6; Experimental: n = 15). [symbol]
Dementia status was unknown in a subset of the patients (Control: n = 10; Experimental: n = 9). [symbol]
indicates data missing or illegible when filed

1. A method for preventing patient falls in a care home, comprising:
exposing subjects in a care home to a lighting system that:
a) in patient rooms, provides blue-enriched white light during daytime hours and low-intensity, blue-depleted white light during nighttime hours;
b) in common areas, provides graduated high-intensity, blue-enriched white light during daytime hours, low-intensity, blue-depleted white light during evening hours, and lower-intensity, blue depleted white light during nighttime hours; and
c) in activity areas, provide high-intensity, blue-enriched white light during activity hours.

2. (canceled)

3. (canceled)

4. The method of claim 1, wherein said patient rooms are provided at least five distinct lighting periods: an early morning period, a mid-morning and afternoon period, an early evening period, an evening period, and a bedtime period.

5. The method of claim 4, wherein light fixtures in said patient rooms generate a minimum of 40 mel-EDI during said early morning period.

6. The method of claim 4, wherein light fixtures in said patient rooms generate a minimum of 100 mel-EDI during said mid-morning and afternoon period.

7. The method of claim 4, wherein light fixtures in said patient rooms generate a maximum of 40 mel-EDI during said early evening period.

8. The method of claim 4, wherein light fixtures in said patient rooms generate a maximum of 20 mel-EDI during said evening period.

9. The method of claim 4, wherein light fixtures in said patient rooms generate a maximum of 10 mel-EDI during said bedtime period, under control of a patient.

10. The method of claim 9, wherein, under medical staff control, said patient rooms can switch during said bedtime period, to generate a maximum of 100 mel-EDI.

11. (canceled)

12. (canceled)

13. The method of claim 1, wherein said common areas are provided at least six distinct lighting periods: an early morning period, a mid-morning period, an afternoon period, an early evening period, an evening period, and a bedtime period.

14. The method of claim 13, wherein light fixtures in said common areas generate a minimum of 100 mel-EDI during said early morning period.

15. The method of claim 13, wherein light fixtures in said common areas generate a minimum of 135 mel-EDI during said mid-morning period.

16. The method of claim 13, wherein light fixtures in said common areas generate a minimum of 100 mel-EDI during said afternoon period.

17. The method of claim 13, wherein light fixtures in said common areas generate a minimum of 60 mel-EDI during said early evening period.

18. The method of claim 13, wherein light fixtures in said common areas generate a minimum of 30 mel-EDI during said evening period.

19. The method of claim 13, wherein light fixtures in said common areas generate a maximum of 10 mel-EDI during said bedtime period.

20. The method of claim 1, wherein said activity areas comprise dining areas and recreational activity areas.

21. The method of claim 20, wherein light fixtures in said dining areas generate a minimum of 135 mel-EDI when occupied and are otherwise off.

22. The method of claim 20, wherein light fixtures in said recreational activity areas generate a minimum of 150 mel-EDI when occupied and are otherwise off.

23. The method of claim 1, further comprising: d) in staff areas, providing high-intensity, blue-enriched white light.

24. The method of claim 23, wherein said staff areas comprise nursing stations/control rooms and staff break rooms.

25. The method of claim 24, wherein light fixtures in said nursing stations/control rooms generate a minimum of 150 mel-EDI at all time periods.

26. The method of claim 24, wherein light fixtures in said staff break rooms generate a minimum of 250 mel-EDI when occupied.

27. (canceled)

28. (canceled)

29. A method for improving resident well-being and staff performance in a facility, comprising: exposing residents and staff in a facility to a lighting system that:
a) in resident rooms, provides blue-enriched white light during daytime hours and low-intensity, blue-depleted white light during nighttime hours;
b) in common areas, provides graduated high-intensity, blue-enriched white light during daytime hours, low-intensity, blue-depleted white light during evening hours, and lower-intensity, blue depleted white light during nighttime hours; and
c) in activity areas, provide high-intensity, blue-enriched white light during activity hours; and
d) in staff areas, provide high-intensity, blue-enriched while light during all hours, when occupied.

30.-56. (canceled)
